(19) United States
(12) Patent Application Publication
(10) Pub. No.: US 2011/0104822 A1
(43) Pub. Date: May 5, 2011

(54) Determination of Distribution

(76) Inventors: Maria Lönnberg, Knivsta (SE); Jan Carlsson, Uppsala (SE)

(21) Appl. No.: 12/999,438

(22) PCT Filed: May 27, 2009
(86) PCT No.: PCT/SE09/00271

§ 371 Date: Dec. 16, 2010

Related U.S. Application Data
(60) Provisional application No. 61/073,614, filed on Jun. 18, 2008.

(30) Foreign Application Priority Data
Jun. 19, 2008 (SE) 0801438-3

(51) Int. Cl. G01N 33/566

(52) U.S. Cl. 436/501

(57) ABSTRACT

A method for determining the occurrence of an analyte subpopulation of heteroforms of a substance (=S) in a liquid sample. The method comprises as its main characteristic features the step of: (i) providing a flow path which a) comprises an outlet part and an inlet part, b) comprises a capture zone (CZ) containing a solid phase exhibiting an immobilized analyte specific binder (B) [=affinity counterpart to the substance] which is capable of affinity binding to S with an affinity that differs for the various heteroforms of S, and c) permits capillary suction from the outlet part for driving a liquid flow through CZ, (ii) flowing said liquid sample containing S in the downstream direction through CZ while S is captured by said binder B in CZ, (iii) determining the distribution of S along the flow direction in CZ by measuring the relative amount of S in at least one subzonei of CZ, and (iv) determining the occurrence of the analyte subpopulation based on the distribution determined in step (iii).

### TECHNICAL FIELD

The present invention relates to a method for determining the occurrence of a subpopulation of heteroforms of a substance (=S) which is present in a liquid sample containing also other heteroforms of S. The subpopulation to be determined is also called analyte (=analyte subpopulation). The method may be used for
a) the diagnosis and/or monitoring of a disease associated with a changed level of a particular subpopulation of S in a body fluid of an individual having or being suspected of suffering from the disease,
b) monitoring an individual's use of a bioactive compound leading to a changed level of a particular subpopulation of S in a body fluid of the individual, and/or
c) monitoring the production of a bio-organic substance S which shall comprise a particular composition or subpopulation of heteroforms of S, e.g. by cell culturing, tissue culturing etc.

Monitoring in (b) includes that the bioactive compound is used for creating a biological response in a living individual, e.g. as a medication, an abuse, production of a bio-organic compound in the individual etc. Immunizations both as part of a therapeutic treatment or for producing antibodies are included.

Patents and patent applications cited in this specification are hereby incorporated in their entirety by reference.

### DEFINITIONS

Heteroforms are variants of a substance and are capable of affinity binding to a common affinity counterpart. Typically the binding is taking place in an inhibitive manner, i.e. the binding of a heteroform to the affinity counterpart is inhibited by one or more of the other heteroforms of the substance such as by competition for the same binding site. Heteroforms may be isoforms of proteins, e.g; isoenzymes, antibodies or immunoglobulins (Igs) of different classes, subclasses, antigen specificities, epitope specificities etc. The heteroform concept also includes that the substance is a bioaffine complex with the individual heteroforms being complexes between a common affinity counterpart and various heteroforms of a protein or the like. Specific examples are immune complexes for which a) the antigen is common but the antibodies are different (e.g. different with respect to class, subclass, epitope specificity etc), and/or b) the antibody is common (e.g. monoclonal) and the antigen comprises heteroforms (e.g. by being polymorphic). One way of determining of whether two variants of a substance are heteroforms to each other are by performing so called inhibition tests.

In its most generic context the term “subpopulation” means a single heteroform or a combination of two or more heteroforms of a substance. In the context of the invention the term “the subpopulation to be determined” typically refers to heteroforms having a common origin, for instance a) produced in a certain organ of a living body or a particular kind of host cell by recombinant techniques, b) occurring in a living body as a consequence of a particular disease, intake of a drug or any other bio-active substance or compound, either therapeutically or as an abuse. Various kinds of recombinantly produced forms of a protein are then also considered as separate subpopulations, e.g. differently mutated variants, variants having the same polypeptide backbone but produced in different kinds of cells. Heteroforms may be common for more than one subpopulation. The subpopulation to be determined thus occurs in the liquid sample together with one or more subpopulations having other origins of the kinds referred to above.

Individual subpopulations to be determined in the invention are typically characterised in containing one or more particular heteroforms of a substance in elevated or decreased amounts relative to a) the total absolute amount of the substance, and/or b) the total absolute amount of a combination of one or more other heteroforms.

The terms level, amount and concentration are used interchangeable and refer to either absolute or relative/normalized values although if not otherwise indicated they primarily refer to normalized values, i.e. an absolute value related to a standard value that normally is the total amount of analyte in the sample concerned.

### BACKGROUND TECHNOLOGY

Most analytes of interest for the invention are heteroforms of circulating glycoproteins which is a class of compounds that in vivo often are extremely heterogeneous with respect to content of heteroforms. This in combination with the fact that the heteroform content of individual subpopulations typically are overlapping, i.e. heteroforms may be common for several subpopulations, has made it problematic to utilize separations based on subtle structural differences to reliably distinguish the occurrence of a particular subpopulation in a parent biological sample containing also other subpopulations of the same glycoprotein.

For erythropoietin (EPO), for which the estimated number of heteroforms is about 30-50, the prior art methods have utilized differences in sialyl group content and comprised a first step in which EPO of a sample, e.g. a urine or a serum sample, is concentrated followed by electrophoresis, in particular isoelectric focusing (IEF), of concentrated EPO in order to separate heteroforms from each other. Labelled lectins have been suggested to be used for the determination of deviations in the isoform pattern found. An important purpose has been to find heteroform pattern deviations that reflect the occurrence of particular abnormal subpopulations and heteroforms which a) derive from exogenous EPO that has been administered to an individual, e.g. various recombinant forms, or b) are disease-related. See further our copending international patent application WO 2008153462 “Determination of a subpopulation of isoerythropoietins” filed on Apr. 21, 2008 and publications cited therein.

Various chromatographic techniques based on affinity principles, such as ion exchange and/or other kinds of affinity, have been suggested to be used in diagnostic assays for clinically determining analyte subpopulations:
a. Column chromatography for i) detecting alcohol abuse by detecting transferrin subpopulations in eluates (Cervén E et al., WO 1982000204; Joustra et al., WO 1985003758 etc), and ii) grading polyclonal antibody responses according to presence of subpopulations (Gyros A B, WO 20060009505) by detecting isoform pattern across the column, possibly in combination with measuring also in eluate fractions in order to generate useful information.
b. Lateral chromatography in a porous sheet material and measurement of particular subpopulations by measuring EPO in a detection zone located downstream of a separation zone for diagnosis of EPO-related diseases and/or abuse of EPO (Carlsson, J & Lönnberg, M, U.S. Pat. No. 6,902,889, U.S. Pat. No. 6,737,278, U.S. Pat. No. 6,528,322, US 20040023412 and our copending international patent application WO 2008153462 “Determination of a subpopulation of isoerythropoietins” filed on Apr. 21, 2008;

Immunoassays in flow matrices for the determination of isoforms have been described in Maria Lönnberg “Membrane-Assisted Isoform ImmunoAssay: Separation and Determination of Protein Isoforms” Thesis 2002, Uppsala University and in publications discussed therein.

In a search report compiled by the SE patent office in the SE priority application the following additional documents have been cited: A) Lönnberg et al., J. of Immunol. Meth. 246 (2000) 25-36; B) Lönnberg et al., J. Chromatog. B 763 (2001) 107-120; C) Lönnberg et al., Anal. Biochem. 293 (2001) 224-31, and EP 0724157 (Bayer Corporation). Documents (A), (C) and EP 0724157 relate to the determination of the total absolute amount of an analyte in a capture/detection zone in which there is no need for using a binder/capturer that is capable of discriminating between different heteroforms of the analyte.

The assays so far approved for clinical use within the field of the invention are time-consuming, expensive and require highly trained and specialized laboratories. There is a need for simplified assays. Complications have been the complex isoform patterns that are obtained for endogenous as well as for recombinantly produced glycoproteins. Due to overlapping, the complexity is enhanced if recombinantly produced variants are present together with endogenous variants, such as in samples deriving from individuals to which recombinant variants have been administered therapeutically or as a doping agent. Metabolization of administered exogenously created variants will further complicate the situation. It has been considered more or less impossible to base reliable clinical assays on chromatographic techniques separating isovariants from each other for a reliable qualitative and quantitative determination of a clinically relevant subpopulation in a parent sample containing also other subpopulations, e.g. disease-related or non-endogenous variants. The similarity of various isoforms and the isoform complexity render it difficult to allow for simple immunoassays of a certain subpopulation in the presence of other subpopulations.

### OBJECTIVES

The main objectives of the invention are to provide methods within the field defined under the heading “Technical Field” above overcoming at least partly one or more of the shortcomings discussed under the heading “Background Technology”.

### INVENTION

The present inventors have recognized that the objectives can be met by a method as defined under the heading “Technical Field” comprising capturing the analyte together with other heteroforms of the substance (=S) in a flow path which
a) comprises an outlet part and an inlet part,
b) comprises a capture zone (CZ) containing a solid phase exhibiting an immobilized analyte specific binder (=B) which is capable of affinity binding to S with an affinity that differs for the various heteroforms of S, and
c) permits capillary suction from the outlet part for driving a liquid flow through CZ,

In addition to the step of providing this kind of flow path (=step (i)), the inventors have recognized that the method should comprise at least the steps of:
(ii) flowing a liquid sample containing S in the downstream direction through CZ while S is captured by said binder B in CZ,
(iii) determining the distribution of S along the flow direction in CZ by measuring the relative amount of S in at least one subzone₁ of CZ, and
(iv) determining the occurrence of the analyte subpopulation in said liquid sample based on the distribution determined in step (iii).

The at least one subzone₁ in step (iii) have been selected so that the occurrence of the analyte subpopulation in the sample give rise to a characteristic pattern of relative amounts in these at least one subzone₁ (=distribution).

For substance S and/or analytes that exist at concentrations below 10⁻⁷ M in a parent sample and/or in the sample used in step (ii) advantageous and surprising results are in particular obtained for labels and detectors that in this specification are indicted to be selected in front of others (i.e. indicated by terms such as “should”, “advantageous”, “preferred”, “in particular” and the like).

The reduction to practice of the invention is clearly showed in the experimental part. The occurrence in a sample of a subpopulation of heteroforms of a substance S occurring in the sample is determined based on the amount of substance S in a subzone₁ of the capture zone relative to the amount of substance S in a standard subzone of the same capture zone.

Flow Path and Flow Arrangements (Steps (i) and (ii))

The flow path is typically defined in a flow matrix placed a) as a surface layer on, or b) fabricated in the surface layer of a planar substrate, and supports capillary transport of an aqueous liquid for the transportation of aqueous liquid samples containing S and/or reagents through CZ. A flow matrix typically is defined as i) a single flow channel including its inner surface (for instance a channel of capillary dimension and with wettable surface characteristics), and ii) a matrix having a penetrating system of hydrophilic flow channels (porous matrices). A flow matrix may be in the form of a porous monolith, porous sheet, column, separate flow channels, or an aggregated system of flow channels. It may also be in the form of particles packed in column cartridges or in cut grooves, compressed fibres etc. The flow path is in preferred variants defined in a flow matrix in the form of a hydrophilic adsorbent sheet material placed on an inert substrate or backing. The substrate/backing is typically hydrophobic and/or impermeable for the liquids used. The flow path may in these and other variants be covered with a lid which is impermeable for the liquid, or uncovered.

The flow matrix shall provide sufficiently small microstructure dimensions in combination with inner surface characteristics of sufficient wettability for an aqueous liquid to be transported into the flow path or matrix by capillarity (self-suction) when the liquid is placed in liquid contact with the inlet part of the flow path. Thus the flow path used in the invention in a first main alternative may be designed as a laterally extending microstructured surface area in a planar material, for instance a) as one or more laterally extending grooves or microchannels and/or b) comprise microprojections extending substantially perpendicular to the surface and at a sufficiently short distance from each other to provide self-suction of a hydrophilic liquid such as water which is placed in liquid contact with an inlet part of the flow path. Typical values for sufficient wettability are water contact angles ≦90°, such as ≦45° and preferably ≦30° (measured at the temperature of use). See for instance WO/2007/149043, WO 2007/149042, 2006/137785, WO 2005/118139; WO 2005089082 (all of Åmic AB). A second main alternative which is preferred means that the microstructured surface area is a hydrophilic porous adsorbent sheet material placed on a backing in the form of a planar substrate which is impermeable for the liquid to be transported in the flow path. See for instance U.S. Pat. No. 6,902,889, U.S. Pat. No. 6,737,278, U.S. Pat. No. 6,528,322, US 20040023412, and our copending international patent application WO 2008153462 “Determination of a subpopulation of isoerythropoietins” filed on Apr. 21, 2008. In hybrid variants the flow path comprises various sections each of which is according to either (a) or (b) of the first alternative, or according to the second alternative. In preferred variants at least the solid phase of the capture zone (CZ) is according to either (b) of the first alternative or according to the second alternative. Other specific alternatives comprise that the flow path is defined by a single microchannel/groove or aggregated microchannels/grooves and that CZ is a section of the flow path in which the microchannels contains packed particles.

The flow path is typically part of a device containing an application zone (AZ) for a) the liquid sample containing S to be flowed through CZ, or b) a liquid sample to be processed within the device to this kind of sample. The liquid sample according to b) may for instance contain heteroforms in addition to those that are to be passed through CZ in which case there may be a separation zone (SZ) located between AZ and CZ in order to remove such additional heteroforms (Carlsson & Lonnberg, WO 9960402, WO 0111355, WO 0111363 and US patents and patent applications deriving therefrom). The device provides for liquid communication between AZ and the inlet part of the flow path containing CZ. This liquid communication is in the simplest variant designed as a flow path as described for (a) or (b) of the first alternative or as described for the second alternative (see above).

The inlet part of the flow path containing CZ is typically also capable of being placed in liquid communication with a reservoir for liquid used for creating a continuous liquid flow through the flow path before or after a sample containing S or a reagent of the type described below has been introduced into the flow path. Similarly the outlet part of the flow path containing CZ is typically capable of being placed in liquid communication with a reservoir for collecting liquids having passed through CZ. This collecting liquid reservoir is typically in the form of a hydrophilic adsorbent such that once the flow channel is filled with an aqueous liquid and liquid communication established between this reservoir and the upstream liquid storage reservoir via the flow path, a capillary driven suction is established creating a liquid flow from the inlet part of the flow path, through CZ, to the outlet part of the flow path. These inlet and outlet parts of the flow path containing CZ may be functionally interchangeable, i.e. the part used for inlet of sample may be the outlet part for liquids passing through CZ when another liquid, such as a reagent liquid, a desorption liquid or a washing liquid, is allowed to enter the flow path (reversing flow direction through CZ). The liquid reservoir for collecting liquids that have passed through CZ is typically at this stage of these variants of the inventive method replaced with a reservoir containing fresh liquid, for instance a desorption or a washing liquid.

One kind of suitable flow matrices have liquid contact surfaces which expose carbohydrate structures, such as cellulose structures, to a through-passing liquid. This kind of surface structures may in preferred variants contain nitro groups such as in nitro cellulose. Suitably matrices typically have pore sizes within the interval of 0.5-15 μm, with preference for the interval of 3-10 μm. The flow matrix in which the flow path containing CZ is defined should be capable of supporting a flow rate in the interval of ≦5 cm/min, such as ≦2.5 cm/min and preferably ≦1 cm/min or ≦0.5 cm/min by the capillary suction discussed herein and/or with a HETP (Height Equivalent of Theoretical Plate) in the interval of ≦50 μm, such as ≦20 μm and with a typically lower limit of 5 μm or 10 μm. These intervals refer to the kind of pore sizes, flow rates, and HETP referred to in Maria Lönnberg “Membrane-Assisted Isoform ImmunoAssay: Separation and Determination of Protein Isoforms” Thesis 2002, Uppsala University.

The capillary driven liquid flow described in the preceding paragraph is typically used for transporting a) the liquid sample containing S and/or b) a liquid aliquot/sample containing an analytically detectable reagent for detecting and measuring S captured in CZ and/or c) as already indicated liquids for washing and/or desorption.

### Substance S and the Analyte

Substance S is preferably a bio-organic macromolecule or biopolymer comprising one or more structures selected amongst carbohydrate/polysaccharide structures, nucleotide/polynucleotide structures, peptide/polypeptide structures, and lipid structures. The various heteroforms of a substance S differ from each other with respect at least one of these structures and include:
A) natively produced variations in amino acid sequence,
B) posttranslational modifications, such as deamidation, addition of carbohydrate structures, phosphorylation, sulphonation etc,
C) modifications by recombinant techniques for replacement, deletion and/or addition of one or more amino acid residues (=protein analogues),
D) chemical modifications, e.g. fragmentation and other derivatizations such as conjugation,
E) number of equal or different subunits which are non-covalently associated to each other (monomer and multimers, such as dimer, trimer etc), and/or
F) variation in charges, for instance in total charge (net charge).

Preferred as substance S are biopolymeric compounds that exhibit polypeptide structure.

The term polymeric structure is generic and thus includes oligomeric structures as well as truly polymeric structures.

Substances existing in the liquid sample used in step (ii) or in a parent sample at a concentration of ≦10⁻⁷ M, in particular ≦10⁻⁹, are of particular interest to be used as substance S in the invention. These limits are also applicable to the analyte.

Examples of potential substances/variations that can be used in the inventions are:
A) Glycoproteins each of which comprises heteroforms differing from each other as discussed above, e.g. with regard to carbohydrate contents (glycosylation) and/or with the same or a similar polypeptide backbone.
   a) Heteroform/isoform variations for glycoproteins are known in a number of diseases, such as cancer, inflammation, liver diseases etc. Particularly may be mentioned the measurement of i) combinations of asialo, monosialo- and disialotransferrins, ii) HbAlc (subpopulation of hemoglobin), iii) subpopulations of erythropoietin etc.
   b) Variations in the carbohydrate contents of glycoproteins are known for normal biological changes, e.g. during the menstruation cycle, during the life time of a person, between males and females etc.
   c) Variations in the degree of glycosylation are known to occur during the production of recombinant proteins depending on conditions utilized, fermentation time etc.
B) Heteroform/isoform variations of enzymes are known to reflect activity.
C) Heteroform/isoform variations of receptor-binding proteins, peptides and other biomolecules are known to influence capability of binding to the receptor (for instance full, reduced or no capability).
D) Heteroform/isoform variations for proteins, peptides and other biomolecules are known to influence strength in affinity towards their affinity counterparts.
E) Heteroform/isoform variations in native transport proteins for exogenous substances (e.g. drugs) or endogenous substances may relate to number of exogenous or endogenous substances bound to the transport protein. Serum albumin is a typical transport protein for drugs. Thyroxine-binding globulin (TBG) and thyroxine-binding prealbumin (TBPA) are transport proteins for triiodothyronine and thyroxine.
F) Heteroform/isoform variations reflected as changed properties of IgG and/or IgA are known for rheumatic or autoimmune diseases.
G) Heteroform/isoform variations reflected as the presence of different degradation fragments of a parent protein are known for certain proteins. Degradation of creatine kinase, for instance, leads to heteroforms/fragments that can be used as markers of cardiac diseases.
H) Heteroform/isoform variations in mixtures of different antibodies are known to reflect the efficiency of the mixture. The mixture may contain different antibodies directed towards the same antigen or the same binding site on an antigen, for instance an antigen-specific polyclonal antibody response or a mixture of monoclonal antibodies containing antigen/hapten specific antibodies. The mixture may contain antibodies of IgA-, IgG-, IgD-, IgE- and/or IgM-class/subclass.

The examples of substance S and analytes given above are adapted to the invention by immobilizing the appropriate B in CZ as outlined in this specification. Further advices about how to arrange the assays may be found in WO 9960402 (Carlsson & Lönnberg) and also in WO 20060009505 (Gyros A B). Alternative H may for instance utilize the appropriate antigen/hapten/allergen as B in CZ. The use of a IgA-, IgG-, IgD-, IgE- and/or IgM-specific analytically detectable reagent in step (iii) are then likely to enable the determination of subpopulations of antibodies of different classes/subclasses and/or levels of binding ability (affinity) for the antigen/hapten/allergen. As described in WO 20060009505 this may be used in grading an immune response in an individual, for instance for grading an IgE mediated allergy (allergen as B in CZ for step (ii) and analytically detectable anti-IgE for step (iii).

### Samples Containing the Analyte and Other Heteroforms of Substance S

The liquid sample used in step (ii) is typically a sample a) of a biological fluid containing a substance that is present in heteroforms and capable of defining an analyte as described in this specification, or b) derived from a parent sample of this kind of biological fluid. Typical such fluids are body fluids for instance whole blood and various blood fractions such as plasma and serum, urine, lachrymal fluid, cerebrospinal fluids, intestinal fluid, etc, cell culture supernatants, supernatants from homogenised tissue or cells etc or any other fluid containing a bioorganic substance which in particular shall comprise one or more of the structures discussed for S above. A parent sample may be processed within and/or outside the device to a sample adapted to be handled within the flow path containing CZ. Device internal or device external processing of the parent sample and of any intermediate liquid sample typically comprises decreasing the level of non-analyte components adversely affecting the measurement of S. If S is a glycoprotein, for instance, and B has specificity for a carbohydrate structure on S it may be beneficial to transfer a parent or an intermediate sample to a sample deficient in glycoproteinic non-analyte components, e.g. by transforming the sample by affinity adsorption to a sample specifically enriched in S. This kind of processing may also include transferring a sample to a sample having an increased relative or absolute concentration of heteroforms that are characteristic for the analyte subpopulation. Processing within the device may include processing in a flow path containing a separation zone (SZ) and desorption from SZ by a liquid flow that is of the same, transversal or opposite direction as the flow utilized for passing the sample containing the analyte through SZ. This desorption flow typically is in downstream liquid communication with the inlet part of the flow path containing CZ. Devices with flow paths utilizing SZ and a combined capture and detection zone (CZ/DZ) with liquid desorption flow from SZ for transport into CZ/DZ have been described previously. See for instance our copending international patent application (“Determination of a subpopulation of isoerythropoietins” international patent application WO 2008153462) and publications cited herein.

### The Capture Zone, Solid Phase and Analyte Specific Binder (B)

The capture zone (CZ) is defined as the section of the flow path between the most upstream and the most downstream position containing immobilized binder (=B). The solid phase is typically a flow matrix which is selected amongst the kinds of flow matrices discussed above with preference for being of the same general type of material as the material in which the flow path is defined.

CZ is typically in the form of a single line across the flow path. The width of the line, i.e. the extension in flow direction, is typically within the interval of ≦10 mm, such as ≦5 mm or ≦mm with preference for ≦1 mm or ≦0.5. As a rule the width is typically ≧0.1 mm, such as ≧0.25 mm. In relation to the detector used for measuring in step (iii) the width should allow for two, three, four or more edge-to-edge placed pixels, with preference for ≧10, such as ≧15 or ≧25 such pixels. In the case of capturing zones which comprises several lines of B these figures refer to the sum of the width of the individual lines.

B is in preferred variants present in immobilized form in CZ from the upstream end to the downstream end of CZ with no segments of CZ being devoid of B. The concentration of B in CZ may vary between different positions in CZ, for instance by having a constant, an increasing or a decreasing concentration in the downstream direction of CZ. The capacity of CZ for capturing S is typically sufficiently high for essentially 100% capture of the amount of S passing into CZ at the flow rates and other conditions provided by the S-carrying liquid (=excessive capacity in CZ). This does not exclude that there may be benefits with arranging B as two or more segments with no or a very low concentration of B between the segments and with a deficient capacity in individual segments to bind 100% of analyte isoforms entering a segment. Deficient capacity in this context means capacity of binding ≦50%, such as ≦25% or ≦15% of the amount of analyte in the sample entering CZ at the flow rates and other conditions applied. In this latter variant the capacity for binding S and/or the concentration of B in neighbouring segments may be constant, decreasing or increasing in the downstream direction. Typically numbers of segments in these variants of CZ are ≦15, such as ≦10 or ≦5 or ≦3. The total capacity across all such segments of CZ should be in excess as described for non-segmented CZ.

One can also envisage variants in which S is not captured to essentially 100% at the flow rates use (deficient capacity of binding S).

Essentially 100% capture above means that minor amounts of S may pass through CZ, for instance 0-10%, i.e. the true capture is 90% such as 95% or 100%.

The analyte specific binder B is an affinity counterpart to S and is thus capable of affinity binding to essentially all of the various heteroforms of S which are present in the analyte-containing sample entering CZ. B is selected to have an affinity that differ for different heteroforms making it possible to discriminate a sample containing a changed level of the analyte subpopulation relative to a standard level by measuring relative amount of S in the above-mentioned at least one subzone₁ (=determining a change in distribution) as discussed elsewhere in this specification. In variants believed to be preferred this typically means that B should be capable of discriminating a heteroform or a combination of heteroforms that are characteristic for the analyte subpopulation and measured in one or more of the at least one subzone₁ from heteroforms that are present in other subzones, e.g. in a standard subzone. With respect to S that are glycoproteins this means that B preferentially should be directed towards one or more epitopes that are related to structures that are essentially constant between the heteroforms but still affected by structures that may vary between at least some of heteroforms, e.g. between an analyte heteroform and non-analyte heteroforms. Suitable Bs thus should be directed towards epitopes related to constant parts of the amino acid sequence and/or essentially constant carbohydrate structures of a glycoproteinic S.

B may be a mixture of different binder molecules having different affinity, including for instance specificity for different heteroforms. Mixtures may be beneficial for accomplishing efficient capture of various heteroforms of S.

Preferred Bs are antibodies in which are included antigen/hapten-binding fragments of full length antibodies and various man-made antigen/hapten-binding derivatives and other constructs thereof such as mutated forms, recombinant forms, chimeric forms, single-chain forms and other forms having the desired specificity and affinity for functioning in the invention as B in CZ. Lectins, e.g. native lectins or modified variants thereof, of the appropriate specificity and affinity may also be useful as B. In the context of the invention lectins also include antibodies directed towards carbohydrate structures.

The techniques for immobilization may be selected amongst those that are known in the field, for instance via covalent bonds, affinity bonds (for instance biospecific affinity bonds), physical adsorption (mainly hydrophobic interaction) etc. Examples of bioaffinity bonds that can be used are bonds between individual members of a bioaffinity pair such as avidin/streptavidin/neutravidin etc and biotin or biotin derivatives, a high affinity antibody and a hapten or a derivative of the hapten, etc where one member of the pair is linked to the solid phase and the other to the binder. Examples of other affinity bonds are between polar groups or charged groups on the solid phase and polar groups or charged groups on the binder (includes electrostatic bonds), between hydrophobic groups on the solid phase and hydrophobic groups on the binder. If the appropriate immobilizing affinity group is not inherently present on the solid phase or B, such a group may be introduced by derivatization (chemically, recombinantly etc).

Many times it is advantageous to immobilize B to the solid phase via a carrier molecule to which one, two, three or more molecules of B are covalently attached (per carrier molecule). The carrier molecule may inherently contain the groups that are necessary for its immobilization to the solid phase or is derivatized to contain such groups. These groups may provide for immobilization via covalent bonds or affinity bonds of the types discussed in the preceding paragraph. In preferred variants the bonds between B and the carrier are covalent while affinity bonds are utilized for attaching the carrier to the solid phase. The carrier typically comprises polymer structure and provides multipoint attachment to the solid phase simultaneously with being a carrier for two or more molecules of B (per carrier molecule). Suitable carriers shall be inert towards the intended reaction, i.e. the affinity reaction between substance S and B, and may comprise polypeptide structure, e.g. be an albumin such as serum albumin, or comprise other kinds of polymer structure, e.g. exhibiting a plurality of hydroxyl and/or amide and/or amine groups and if required derivatized to exhibit affinity groups of the types discussed above.

Determining the Distribution (Step (iii))

The determination of the distribution in the CZ comprises measurement of the relative amount of analyte in at least one subzone₁ of CZ.

A subzone₁ may be a single position along the flow direction in CZ or comprise a segment between an upstream and a downstream position in CZ. The at least one subzone₁ has been selected so that a certain found distribution measured as a single relative amount of S for a subzone₁ or a combination of relative amounts for at least two of said at least one subzone₁ will be indicative of the occurrence or non-occurrence of the analyte subpopulation in the sample. As a rule a subzone₁ should not cover the full length of CZ although one can envisage such variants when S is not completely captured in CZ (deficient capacity under the conditions used).

Single position in this context typically means that the subzone corresponds to a width in the flow direction of a pixel.

Measuring the relative amount of S in a subzone₁ comprises that the absolute amount of S present in the subzone is normalized relative to the amount of a standard component. The standard component is preferably the absolute amount of S present in a subzone (standard subzone) which is different from the particular subzone₁ for which the relative amount is calculated. The standard subzone may be a subzone₁ or some other subzone of CZ. A standard subzone is either non-overlapping or overlapping but not completely coinciding with subzone₁ for which the relative amount is to be calculated. A standard subzone may be a single position in CZ or cover a certain length up to the full length of CZ (in the flow direction). In the case the standard subzone covers the full length of CZ normalization will be against the total amount of S in the sample used in step (ii). Preferably the standard subzone is the position of highest relative amount/concentration of S in CZ along the flow direction or comprises this position. For variants in which the binder is an antibody having the preferred specificity given above, this position typically is located to the most upstream position or part of CZ with its content of S typically being a function of the amount of analyte in the parent sample and liquid sample. As an alternative the standard component may be measured separately, for instance by measuring separately the total amount of S in the parent sample or in the liquid sample used in step (ii).

Subzones, e.g. a subzone₁ or a standard subzone may be continuous or discontinuous.

In preferred variants the measuring in step (iii) typically comprises using an analytically detectable reagent that is capable of affinity binding to S or to B, i.e. is an affinity counterpart to S or to B. Step (iii) may thus comprise the steps of:
a) flowing a liquid aliquot/sample containing an analytically detectable reagent through CZ under conditions permitting capturing of the detectable reagent in CZ, and
b) measuring the absolute amount of said reagent in the at least one subzone₁ discussed above.

The conditions that permit affinity capturing of the analytically detectable reagent in CZ are provided by the liquid aliquot. Between step (ii) and step (a) and/or between step (a) and step (b) there may be washing steps, i.e. one or more steps in which a washing liquid is allowed to pass through CZ

Substance S is in certain variants detectable as such meaning that there is no need for a separate detectable reagent. Step (a) as a separate step can then be omitted. Typical examples are variants in which S is an enzyme and variants in which the actual S to be captured in step (ii) is formed by preincubation with an analytically detectable reagent (see below).

The measuring in step (b) comprises obtaining a signal from the detectable label. The value of the signal obtained for a subzone₁ is a function of both the absolute amount of S and the absolute amount of the detectable reagent in the subzone. Thus the absolute amount of S for a subzone₁ and any other subzone may be derived from standard curves obtained by separately measuring increasing standard amounts of S.

When the detectable reagent is an affinity counterpart to the analyte, the reagent is typically selected amongst the different kinds of candidates mentioned above for B. Precautions are that the specificity of the detectable reagent must be for a binding site on S which is structurally different compared to the binding site utilized by B and/or spaced apart from this binding site. The preferences are otherwise the same as for B. Preferred variants of step (a) with this kind of detectable reagent include that step (ii) and step (iii.a) are separate. In other variants the two steps may coincide, e.g. when S is inherently detectable or when S is preincubated with the detectable reagent within or external to the device. In the case of preincubation the liquid sample entering the flow path containing CZ will contain an affinity complex containing both S and detectable reagent and the complex will be the actual substance S to be captured in step (ii). One can envisage advantages if the affinity to heteroforms which are characteristic for the analyte subpopulation is higher than to other heteroforms of S. If the difference is large enough the determination of total level of S in the sample used in step (ii) may need a separate measurement using a detectable reagent with an affinity better adapted to measure total S. This kind of detectable reagents are typically in the form of conjugates between one moiety, which is an affinity counterpart to the analyte, and another moiety, which provides detectability (=label).

When the detectable reagent is a counterpart to the binder it is preferably a labelled form of S, i.e. a conjugate comprising one moiety which provides detectability (=label) and another moiety which is capable of competing with S about binding sites on B.

Conjugates in the context of detectable reagents encompass native as well as man-made conjugates. Labels that can be conjugated to an affinity counterpart and used in affinity assays are well-known in the field and include a) signal-generating groups, such as members of enzymatic systems, fluorophors, radioactive isotopes, chemiluminophors, etc, and b) affinity labels, such as biotin, hapten and other groups that require other labelled conjugates with affinity for the affinity label used.

The label used should have a relatively high detectability such as ≦100 attomole/mm² or ≦50 attomole/mm², or ≦25 attomole/mm² or ≦15 attomole/mm² or ≦10 attomole/mm². Preferred labels often have a still better detectability for instance ≦1 attomole/mm² or even lower such as ≦0.5 attomole/mm². For molar concentrations in the concentration interval given above the detectability should typically be at least 0.01 or at least 0.05 attomole/mm². Particles are preferred as labels, in particular coloured particles giving a high contrast relative to the flow matrix/solid phase present in CZ. In other words dark particles such as black particles and in particular particles made of and/or containing carbon, such as carbon black. This is in particular is applicable when the flow matrix is white or has some other colour providing good contrast with the signal created by the label. Se further Maria Lönnberg “Membrane-Assisted Isoform ImmunoAssay: Separation and Determination of Protein Isoforms” Thesis 2002, Uppsala University which further inform about preferred agglomerated carbon black particles, such as sp 100.

Measuring in step (iii) typically means that the signal in desired subzones of CZ from the detectable immobilized complex formed in step (iii) or in step (ii) is measured and transformed to relative amounts as outlined above. In preferred variants this comprises measuring by the use of a detector, which is capable of creating an image of CZ (imaging detector) reflecting local variations in concentration of the complex formed/detectable reagent/labelvariation within the CZ area. Typical imaging detectors are adapted to measure the signal from the analytically detectable reagent captured in the various subzones of CZ. They are preferably based on the pixel-concept giving pixel sizes as outlined below and including techniques such as CCD, CMOS etc, and can thus be considered as digital cameras. They may be in the form of scanners.

Suitable imaging detectors typically should be capable of giving pixel sizes corresponding to ≧10 pixels/mm, such as ≧15 pixels/mm or ≧25 pixels/mm with preference for even smaller pixels, such as ≧50 pixels/mm or ≧75 pixels/mm in the flow direction. Upper limits are ≦75 or ≦100 pixels/mm.

The imaging detectors should also be selected to have a suitable resolution with respect to greyscale. For the invention this means that suitable scanners/detectors should have an at least 8 bit greyscale with preference for an at least 10, an at least 12, an at least 14 or an at least 16 bit greyscale, e.g. including a greyscale comprising a number of levels that is in the interval of ≧256, such as ≧1024, or ≧4096 or ≦16384 levels or ≧65536 levels. Se further Maria Lönnberg “Membrane-Assisted Isoform ImmunoAssay: Separation and Determination of Protein Isoforms” Thesis 2002, Uppsala University.

Determination of the Occurrence of the Analyte Subpopulation in the Parent Sample (Step (iv))

In this step the relative amount of S in the at least one subzone₁ mentioned above (=distribution) is compared with the corresponding distribution obtained for a standard composition/sample of isoforms of S processed according to the inventive method under essentially equivalent conditions as used for the sample containing S (including step (ii) and possibly also preprocessing as discussed herein). In the case a deviation is found for the at least one subzone₁, this will be indicative of a change in relative amount (increased or decreased) of the subpopulation(s) for which these subzone(s) have been selected. A non-finding of the deviation is indicative of no difference between the relative amount of the subpopulation(s) in the parent sample and the standard composition.

The sample used in step (ii) is typically derived from an individual to be tested for a change in relative amounts of the subpopulation(s).

The standard composition is typically representative for the corresponding samples derived from normal individuals or from individuals that have changed relative amounts for various reasons. A change or deviation in relative amounts (increased or decreased) compared to normal individuals may be characteristic for individuals:
a) suffering from a disease related to a change in the level of said subpopulation in said parent sample, and/or
b) having taken a bioactive compound promoting a change in the level of said subpopulation in said parent sample.

The bioactive compound promoting the change may have been taken as part of an abuse and/or as part of a therapeutic treatment. The bioactive compound may be a substance S containing the analyte subpopulation or a compound leading to the formation in vivo of the analyte subpopulation.

The determination of distribution and/or classification of a found distribution in relation to standard distributions may advantageously be carried out by the use of a computer program (computer based pattern recognition). The invention thus also provides a computer program for carrying out these operations, a carrier medium loaded with such a program as well as a computer loaded with a carrier loaded with this kind of program.

### BEST MODE

The Best Mode reduced to practice is given in the experimental part. Future modes that are believed to be advantageous are indicated in the descriptive part including the experimental part by preferred/advantageous etc clauses.

### EXPERIMENTAL PART

Further details about selection of the techniques utilized in the experimental part including for instance selection of kind, material, properties etc of flow matrices, selection of labels, etc are given by Maria Lönnberg “Membrane-Assisted Isoform ImmunoAssay: Separation and Determination of Protein Isoforms” Thesis 2002, Uppsala University.

### Example 1

### Test to Distinguish EPO and EPO Analogues by Their Different Affinity to EPO Antibodies Using an Immunochromatographic Test

Sample material: Neorecormon®, recombinant epoetin beta, and MIRCERA®, a methoxy polyethylene glycol-epoetin beta was obtained from Roche Diagnostics GmBH (Mannheim, Germany). Aranesp®, the recombinant EPO analogue darbepoetin, was purchased from Amgen (Thousand Oak, Calif., USA). Dilution series was performed in 20 mM bis-tris buffer, pH 6.5, 0.1 M NaCl, 0.1% Tween 20 and 0.02% NaN₃.

Measurement of EPO concentration and calculation of affinity ratios: The dilution series of EPO and the two EPO analogues were tested by an immunochromatographic EPO test where 25 μl of sample in duplicate was dispensed in microtiter wells and a 5 mm wide and 22 mm long porous lateral flow strip (MAIIA AB, Uppsala, Sweden), with a thin line of anti-EPO 3F6 about 13 mm from one end of the membrane with the other end mounted on a 30 mm absorbent sink, was placed in each well. After 5 minutes the complete sample volume had been sucked up and the strip was moved into another well containing 25 μl of carbon black antiEPO 7D3 (MAIIA AB) in which it was left for 5 minutes and finally placed into a well containing 25 μl washing solution (MAIIA AB) for 5 minutes.

The strips were mounted on a paper sheet, the absorbent sink was removed and the sheet was placed in a scanner after the strips had dried. The intensity of carbon black in the capturing anti-EPO zone was measured for each strip, and delta blackness per pixel (signal−baseline signal) was calculated at the maximal signal (the peak) for the average of 3 rows of pixels (3×42.3 μm) in accordance with earlier description [Anal. Biochem. 293, 224-231 (2002)]. In addition, delta blackness per pixel at several positions down-stream the peak was also calculated using the average of 3 rows of pixels.

A standard-curve was prepared by correlating the known EPO concentration for the dilution sequence of Neorecormon to the corresponding delta blackness per pixel for the peak.

The ratio between the EPO concentration calculated from the peak value and from subsequent positions (0.21 and 0.42 mm) was used to reveal the affinity characteristic of different types of EPO and its analogues.

Results: See Table 1, page 22

### Example 2

### Test to Distinguish EPO and EPO Analogues in Urine by Their Different Affinity to EPO Antibodies Using an Immunochromatographic Test

Sample material: Urine specimens were collected from healthy individuals. Eprex®, recombinant epoetin alpha, Janssen-Cilag AB (Sollentuna, Sweden) and Aranesp®, the recombinant EPO analogue darbepoetin, was applied in a concentration of 25 ng/L to a urine with endogenous EPO below 5 ng/L. The thawed urines were gently turned end-over-end to distribute the precipitates evenly and an aliquot was transferred to another tube together with Urine Precipitate Dissolvation buffer (MAIIA AB), 9 parts urine and one part buffer. The urine precipitates was instantly dissolved and 2.5 ml of the obtained solution was desalted on a PD10 column by elution with 3.5 ml of buffer (20 mM Tris pH 7.5, 75 mM NaCl, 0.1% tween 20 and 0.02% NaN₃). Eprex was used as a standard and a dilution series (0.3-100 ng EPO/L) was prepared in 0.03% BSA, 20 mM TRIS pH 7.5, 75 mM NaCl, 0.1% tween 20 and 0.02% NaN3.

### Measurement of EPO Concentration and Calculation of Affinity Ratios

The procedure was in accordance with Example 1 but 200 μl of desalted urine or Eprex standard was used and the incubations times in each well were 75, 6 and 6 minutes.

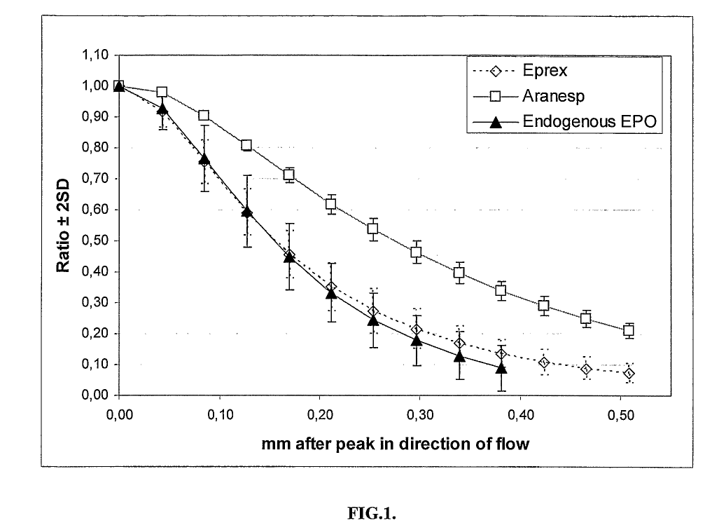

Results: FIG. 1: The calculated ratio for different positions along the anti-EPO zone on the strips shows that the ratio for EPO is different from the ratio for the EPO analogue Aranesp. EPO is represented by six samples where Eprex (recombinant epoetin alpha) was added to urine and by five urines containing endogenous EPO. Six samples contained Aranesp added to urine.

During the priority year there have been indications that it can be advantageous to include measurements in two or more subzones₁ both for samples and standard.

While the invention has been described and pointed out with reference to operative embodiments thereof, it will be understood by those skilled in the art that various changes, modifications, substitutions and omissions can be made without departing from the spirit of the invention. It is intended therefore that the invention embraces those equivalents within the scope of the claims which follow.

| | A | | B | | | C | | |
|---|---|---|---|---|---|---|---|---|
| | dspp | | ratio | | | ratio | | |
| | 0.00 | CV % | 0.21 | CV % | | 0.42 | CV % | |
| Neo 10 ng/L | [symbol] | 0.04 | [symbol] | 1.24 | | [symbol] | 51.87 | |
| Neo 30 ng/L | 2720 | 9.85 | 0.54 | 4.36 | | 0.27 | 1.93 | |
| Neo 100 ng/L | 6781 | 4.88 | 0.54 | 3.09 | | 0.25 | 5.76 | |
| Neo 300 ng/L | 15150 | 0.82 | 0.54 | 0.98 | Mean | 0.25 | 2.80 | Mean |
| Neo 1000 ng/L | 25929 | 1.23 | 0.46 | 0.32 | 0.52 | 0.20 | 0.34 | 0.24 |
| Mir 50 ng/L | [symbol] | 4.57 | [symbol] | 0.23 | | [symbol] | 8.60 | |
| Mir 150 ng/L | 1934 | 4.62 | 0.87 | 3.15 | | 0.74 | 3.00 | |
| Mir 500 ng/L | 5121 | 1.82 | 0.87 | 2.26 | | 0.73 | 1.81 | |
| Mir 1500 ng/L | 13153 | 1.67 | 0.83 | 4.23 | Mean | 0.65 | 2.96 | Mean |
| Mir 5000 ng/L | 24345 | 3.65 | 0.75 | 1.36 | 0.83 | 0.54 | 1.72 | 0.66 |
| A 10 ng/L | [symbol] | 13.46 | [symbol] | 5.19 | | [symbol] | 14.13 | |
| A 30 ng/L | 1974 | 1.89 | 0.74 | 5.74 | | 0.48 | 9.71 | |
| A 100 ng/L | 5753 | 3.89 | 0.75 | 0.83 | | 0.51 | 3.27 | |
| A 300 ng/L | 13277 | 3.65 | 0.74 | 5.03 | Mean | 0.49 | 4.77 | Mean |
| A 1000 ng/L | 22474 | 1.20 | 0.75 | 1.71 | 0.74 | 0.49 | 3.06 | 0.49 |

Strike-through value = below detection limit

The ratio, between the concentration calculated from the maximal signal peak value (A) and the concentration calculated when measuring at position 0.21 (B) and 0.42 (C) mm downstream the maximal signal, differs for EPO:s like Neorecormon (Neo) and for the EPO analogues Mircera (Mir) and Aranesp (A). When comparing the calculated concentration reduction at position 0.21 mm, the EPO signal is reduced to 0.52 while for Aranesp and Mircera the signal is reduced to only 0.74 and 0.83, respectively. This shows that EPO binds rapidly to the immobilised anti-EPO and there are only low amounts left of EPO to migrate to the position at 0.21 mm. The EPO analogues, on the other hand, bind not so rapidly to anti-EPO and a considerable amount is migrating to the positions downstream the peak value.

1-22. (canceled)

23. A method for determining the occurrence of an analyte subpopulation of heteroforms of a substance (=S) in a liquid sample, comprising the steps of:
(i) providing a flow path which
a) comprises an outlet part and an inlet part,
b) comprises a capture zone (CZ) containing a solid phase exhibiting an immobilized analyte specific binder (B) [=affinity counterpart to the substance], typically an antibody, which is capable of affinity binding to S with an affinity that differs for various heteroforms of S, and
c) permits capillary suction from the outlet part for driving a liquid flow through CZ,
(ii) flowing said liquid sample containing S in the downstream direction through CZ while S is captured by said binder B in CZ,
(iii) determining the distribution of S along the flow direction in CZ by measuring the relative amount of S in at least one subzone₁ of CZ, and
(iv) determining the occurrence of the analyte subpopulation based on the distribution determined in step (iii).

24. The method according to claim 23, comprising obtaining said relative amount in a subzone₁ by measuring the absolute amount of S in this subzone₁ and normalizing the absolute amount found against a standard component which preferably is the absolute amount of S measured in a standard subzone in CZ which is different from and typically is non-overlapping with said subzone₁ and with preference is or contains the position of highest amount of S along the flow direction in CZ.

25. The method according to claim 23, comprising transporting
a) said liquid sample and/or
b) a liquid aliquot which possibly is a washing liquid or contains an analytically detectable reagent for detection of S captured in CZ during step (ii)
through CZ using said capillary suction for creating said liquid flow.

26. The method according to claim 23, wherein said flow path is defined in a hydrophilic adsorbent sheet material placed on a an inert substrate, which typically is hydrophobic and/or impermeable for the liquids used.

27. The method according to claim 23, wherein the measuring in step (iii) comprises
a) flowing a liquid aliquot containing an analytically detectable reagent through CZ, said reagent being capable of affinity binding to S captured in CZ during step (ii) or to unoccupied binder B remaining in CZ after step (ii), and said aliquot providing conditions required for said affinity binding to take place, and
b) measuring the relative amount of said reagent in said at least one subzone₁.

28. The method according to claim 23, wherein the measuring in step (iii) comprises measuring by the use of an imaging detector based on the pixel concept adapted to measure the analytically detectable reagent.

29. The method according to claim 23, wherein
A) the presence of a change in relative amount, e.g. a lowered or increased level, of said subpopulation in said liquid sample creates a characteristic deviation in said distribution compared to a standard distribution obtained under equivalent conditions for said at least one subzone₁ for a standard composition of heteroforms of said substance, and
B) step (iv) comprises
a) comparing the distribution found in step (iii) with said standard distribution, and
b) taking a finding or a non-finding of said deviation to be indicative of the presence or absence, respectively, of said change in relative amount of said subpopulation(s) in said liquid sample.

30. The method according to claim 23, wherein said liquid sample is derived from an individual to be tested for a change in relative amount of said subpopulation, and that said standard composition is representative for the corresponding samples derived from normal individuals and/or that the presence and/or absence of said change is characteristic of individuals
a) suffering from a disease related to a change in the level of said subpopulation in said parent sample, and/or
b) having taken a bioactive compound promoting a change in the level of said subpopulation in said parent sample.

31. The method according to 30, wherein
a) said liquid sample is derived from an individual to be tested for a change in relative amount of said subpopulation, and
b) said standard composition is representative for the corresponding samples derived from normal individuals,
c) said individual belongs to group (b), and
d) said bioactive compound has been taken as an abuse or as part of a therapeutic treatment.

32. The method according to claim 23, wherein
a) said S exhibits polypeptide structure and/or carbohydrate structure, and said heteroforms differ from each other with respect to either one or both of these two structures, and
b) said binder B has specificity for such a polypeptide structure when it is present in one or more of said heteroforms, and typically is an antibody.

33. The method according to claim 23, wherein the flow path is defined in a flow matrix which a) is in the form of an adsorbent sheet material with pore sizes within the interval of 0.5-15 μm and/or b) supports a HETP within the interval of ≦100 μm.

34. The method according to claim 23, wherein the flow path is defined in a flow matrix, e.g. in the form of an adsorbent sheet material, supporting a flow rate of ≦5 cm/min by capillary suction from the outlet end of the flow path.

35. The method according to claim 23, wherein an imaging detector based on the pixel-concept is used for measuring in step (iii), and that the segment of CZ containing immobilized B has a width in the flow direction of at least two edge-to edge placed pixels.

36. The method according to claim 23, wherein the detectable reagent comprises a label, e.g. in the form of particles with preference for black particles such as carbon particles.

37. The method according to claim 23, wherein an imaging detector based on the pixel-concept is used for measuring in step (iii) has a greyscale providing ≧256 levels.
